(19) United States
(12) Patent Application Publication
(10) Pub. No.: US 2025/0285457 A1
(43) Pub. Date: Sep. 11, 2025

(54) MACHINE LEARNING ENABLED HISTOLOGICAL ANALYSIS

(71) Applicant: Genentech, Inc., South San Francisco, CA (US)

(72) Inventors: Lisa Michelle MCGINNIS, South San Francisco, CA (US); Namrata Srivastava PATIL, South San Francisco, CA (US)

(21) Appl. No.: 19/211,000

(22) Filed: May 16, 2025

Related U.S. Application Data
(63) Continuation of application No. PCT/US2023/080347, filed on Nov. 17, 2023.
(60) Provisional application No. 63/384,364, filed on Nov. 18, 2022.

(51) Int. Cl. G06V 20/69; G06T 7/00; G06V 10/764; G16H 20/00; G16H 50/20

(52) U.S. Cl. CPC G06V 20/698; G06T 7/0012; G06V 10/764; G16H 20/00; G16H 50/20; G06T 2207/30096

(57) ABSTRACT

A method may include applying a cell classification model to identify, based at least on an image of a biological sample, one or more cell types present in the biological sample. The cell classification model may be trained to differentiate between a plurality of cell types including a first cell type whose likelihood of being a macrophage satisfies a threshold and a second cell type whose likelihood of being the macrophage fails to satisfy the threshold. A composition profile for the biological sample may be generated based on the one or more cell types identified in the biological sample. At least one of a disease diagnosis, a disease progress, a disease burden, and a treatment response for a patient associated with the biological sample may be determined based on the composition profile of the biological sample. Related systems and computer program products are also provided.

### CROSS-REFERENCE TO RELATED APPLICATIONS

This application is a continuation of International Application No. PCT/US2023/080347, filed on Nov. 17, 2023, which claims priority to U.S. Provisional Patent Application No. 63/384,364, entitled “Machine Learning Enables Histological Analysis” filed Nov. 18, 2022, the disclosure of which is incorporated herein by reference in its entirety.

### TECHNICAL FIELD

The subject matter described herein relates generally to the digital and computational pathology and more specifically to machine learning based approach to cell type classification.

### INTRODUCTION

Cancerous cells trigger significant molecular, cellular, and physical changes within their host tissues in order to support further growth and metastasis. A tumor is typically a heterogeneous collection of cells that include infiltrating and resident host cells as well as secreted factors and extracellular matrix. On a broader scale, the tumor microenvironment may include the immune cells, endothelial cells, and fibroblasts that are present in the vicinity of cancer cells. There exists a strong correlation between the composition of the tumor, the tumor microenvironment, and the tumor's ability to sustain itself at its primary site, evade immune responses, resist drug intervention, and proliferate to various secondary locations.

### SUMMARY

Systems, methods, and articles of manufacture, including computer program products, are provided for machine learning enabled histological analysis. In one aspect, there is provided a system for identifying one or more cell types present in an image of a biological sample. The system may include at least one processor and at least one memory. The at least one memory may include program code that provides operations when executed by the at least one processor. The operations may include: receiving an image of a biological sample; applying a cell classification model to identify, based at least on the image of the biological sample, one or more cell types present in the biological sample, the cell classification model being trained to differentiate between a plurality of cell types including a first cell type whose likelihood of being a macrophage satisfies a threshold and a second cell type whose likelihood of being the macrophage fails to satisfy the threshold; and generating, based at least on the one or more cell types identified in the biological sample, a composition profile for the biological sample.

In another aspect, there is provided a method for identifying one or more cell types present in an image of a biological sample. The method may include: receiving an image of a biological sample; applying a cell classification model to identify, based at least on the image of the biological sample, one or more cell types present in the biological sample, the cell classification model being trained to differentiate between a plurality of cell types including a first cell type whose likelihood of being a macrophage satisfies a threshold and a second cell type whose likelihood of being the macrophage fails to satisfy the threshold; and generating, based at least on the one or more cell types identified in the biological sample, a composition profile for the biological sample.

In another aspect, there is provided a computer program product for identifying one or more cell types present in an image of a biological sample. The computer program product may include a non-transitory computer readable medium storing instructions that cause operations when executed by at least one data processor. The operations may include: receiving an image of a biological sample; applying a cell classification model to identify, based at least on the image of the biological sample, one or more cell types present in the biological sample, the cell classification model being trained to differentiate between a plurality of cell types including a first cell type whose likelihood of being a macrophage satisfies a threshold and a second cell type whose likelihood of being the macrophage fails to satisfy the threshold; and generating, based at least on the one or more cell types identified in the biological sample, a composition profile for the biological sample.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the identification of cell types associated with low confidence expert annotations, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a digital pathology system, in accordance with some example embodiments;

FIG. 2A depicts a flowchart illustrating an example of a process for identifying one or more cell types present within an image of a biological sample, in accordance with some example embodiments;

FIG. 2B depicts a flowchart illustrating another example of a process for identifying one or more cell types present within an image of a biological sample, in accordance with some example embodiments;

FIG. 3A depicts a flowchart illustrating an example of a process for training a cell classification model, in accordance with some example embodiments;

FIG. 3B depicts a flowchart illustrating an example of a process for generating a training set for training a cell classification model, in accordance with some example embodiments;

FIG. 4 depicts a schematic diagram illustrating an example of a tumor microenvironment, in accordance with some example embodiments;

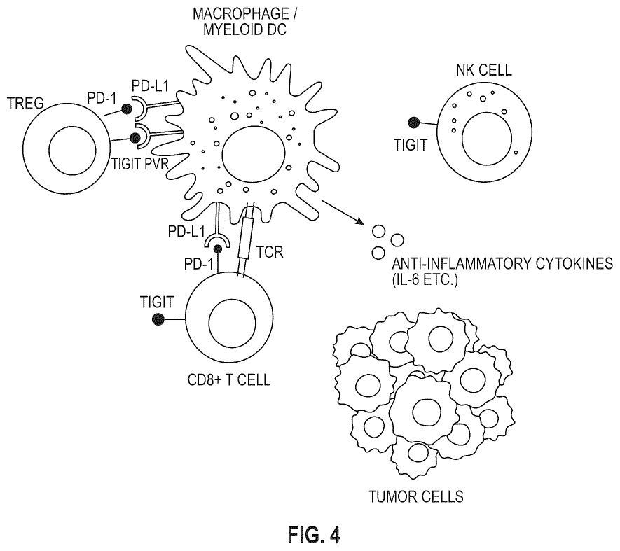

FIG. 5 depicts examples of macrophages associated with high confidence expert annotations, in accordance with some example embodiments; and

FIG. 6 depicts a block diagram illustrating an example of a computing system, in accordance with some example embodiments.

When practical, similar reference numbers denote similar structures, features, or elements.

### DETAILED DESCRIPTION

In highly heterogeneous diseases such as cancer, insights into the types of cells forming diseased tissue and the surrounding microenvironment may be integral to the accurate diagnosis of disease subtype, prognosis of disease progress, and prediction of response to various treatments. For example, non-small cell lung cancer (NSCLC) patients who are likely to respond to combination immunotherapies such as the combination of T-cell immunoreceptor with Ig and ITIM domains (TIGIT) with atezolizumab may be identified based on cancer cells exhibiting a high expression level of the programmed death ligand-1 (PD-L1) gene. Within the population of non-small cell lung cancer patients whose cancer cells are PD-L1 negative or exhibit a low expression level of the PD-L1 gene, further differentiation between responders and non-responders to combination immunotherapies is desired.

The interaction between macrophages and lymphocytes (e.g., CD8 positive T-cells), which triggers release of anti-inflammatory cytokines (e.g., IL-6 and/or the like), may inhibit patient response to combination immunotherapies such as the combination of T-cell immunoreceptor with Ig and ITIM domains (TIGIT) with atezolizumab. Accordingly, macrophages may serve as indicators of treatment response. However, macrophage detection, alone, is not a sufficient indicator. It may be important to know what types of macrophages are present, along with a context of a tumor environment. The present disclosure provides systems and methods for differentiating between responders and non-responders based on the presence of tumor-associated macrophages in the tumor environment. In particular, the present disclosure describes ways to differentiate between low-confidence macrophages and high-confidence macrophages. In addition, the present disclosure presents systems and methods for identifying and distinguishing between various macrophages, including stromal macrophage cells and alveolar macrophages. In classifying macrophages at this level of refinement, the disclosed systems and methods may provide a prediction of treatment response or clinical outcome. In some cases, refined macrophage type classification and detection may serve as a proxy or alternative to gene expression analysis in predicting treatment response.

In some example embodiments, an image depicting a biological sample may undergo histological analysis in order to identify the one or more cell types present therein. For example, in some cases, a machine learning based cell classification model may be applied to a whole slide image of the biological sample (e.g., a hematoxylin and eosin (H&E) stained whole slide image, a multiplex immunofluorescence (MxIF) stained whole slide image, an immunohistochemical (IHC) stained whole slide image, and/or the like) in order to identify one or more tumor cells, lymphocytes, plasma cells, fibroblasts, macrophages, endothelial cells, adipocytes, and neutrophils present in the biological sample. A composition profile for the biological sample may be generated based at least on the one or more cell types identified within the biological sample. At least one of a disease diagnosis, a disease progress, and a treatment for a patient associated with the biological sample may be determined based on the composition profile. For instance, in some cases, the patient associated with the biological sample may be identified as a responder (or non-responder) to a particular treatment (e.g., the combination of T-cell immunoreceptor with Ig and ITIM domains (TIGIT) with atezolizumab) based at least on whether macrophages are identified as being present within the biological sample.

In some example embodiments, the cell classification model may be trained to differentiate between a variety of different cells including, for example, tumor cells, lymphocytes, plasma cells, fibroblasts, macrophages, endothelial cells, adipocytes, neutrophils, and/or the like. In some cases, the cell classification model may be trained based on a training set containing one or more images, each of which being associated with one or more ground truth labels identifying the cell types present therein. Moreover, in some cases, the ground truth labels associated with each image in the training set may be determined based on expert annotations. As such, the performance of the cell classification model may be limited by the accuracy of expert annotations. In instances where the expert annotations fail to accurately identify a particular cell type within the one or more images, the trained cell classification model may perform poorly when encountering that particular cell type. For example, certain types of macrophages, including non-pigmented stromal macrophages, may be difficult to visually decipher in images. Non-pigmented stromal macrophages are often confounded with other stromal cells such as fibroblasts whereas foamy macrophages, alveolar macrophages), and pigmented stromal macrophages are more readily distinguishable. Accordingly, the ground truth labels identifying non-pigmented stromal macrophages and fibroblasts tend to be less reliable. Training the cell classification model to recognize a single monolithic class of macrophages may thus diminish the performance of the trained cell classification model in correctly identifying the macrophages that may be present in a biological sample.

In some example embodiments, the cell classification model may be trained to recognize a first class of macrophages that includes foamy macrophages, alveolar macrophages (including intra-alveolar macrophages), and pigmented stromal macrophages as well as a second class of stromal cells which include the non-pigmented stromal macrophages and fibroblasts. Training the cell classification model to recognize a separate class of stromal cells that include the non-pigmented stromal macrophages likely to be confounded with fibroblasts may increase the performance of the cell classification model in identifying other macrophages including, for example, foamy macrophages, alveolar macrophages (e.g., intra-alveolar macrophages), pigmented stromal macrophages, and/or the like. For example, in some cases, the cell classification model may be trained to differentiate between a plurality of cell types including a first cell type whose likelihood of being a macrophage satisfies a threshold and a second cell type whose likelihood of being the macrophage fails to exceed the threshold. The first cell type whose likelihood of being a macrophage satisfies the threshold may include stromal cells such as fibroblasts and non-pigmented stromal macrophages while the second cell type whose likelihood of being a macrophage fails to satisfy the threshold may include foamy macrophages, intra-alveolar macrophages, and pigmented stromal macrophages. In some cases, in addition to the first cell type and the second cell type, the cell classification model may be further trained to differentiate between tumor cells (e.g., non-small cell lung cancer (NSCLC) tumor cells and/or the like), lymphocytes, plasma cells, endothelial cells, adipocytes, and neutrophils.

In some example embodiments, the cell classification model may be trained based on training data that includes one or more images annotated with ground truth labels identifying at least one cell type present in each image. The uncertainty present in the expert annotation associated with non-pigmented stromal macrophages may be captured in training the cell classification model to recognize a separate class of stromal cells that includes fibroblasts and non-pigmented stromal macrophages. For example, in some cases, the training data may be generated by at least assigning, to the one or more images, a first ground truth label identifying the first cell type (e.g., stromal cells) based at least on a first expert annotation identifying one or more macrophages being associated with a confidence value that fails to satisfy one or more thresholds. Moreover, in some cases, the training data may be generated by at least assigning, to the one or more images, a second ground truth label identifying the second cell type (e.g., macrophages) based at least on a second expert annotation identifying the one or more macrophages being associated with a confidence value that satisfies the one or more thresholds. Accordingly, the ground truth label of an image may indicate the presence of a macrophage if the corresponding expert annotation is sufficiently reliable (e.g., a confidence value satisfying one or more thresholds). Where the expert annotation indicating the presence of a macrophage is insufficiently reliable (e.g., a confidence value that fails to satisfy one or more thresholds), the ground truth label of the image may indicate the presence of a stromal cell instead that of a macrophage.

In some example embodiments, the cell classification model may classify each cell depicted in an image of a biological sample by at least assigning, to each cell, a “hard label” of a specific cell type or a “soft label” of the probability that the cell is positive for each of a plurality of different cell types (e.g., stromal cell, macrophage, tumor cell, lymphocyte, and plasma cell). In some cases, the cell classification model may operate on a segmented image that has undergone segmentation, such as watershed cell segmentation, machine learning based cell segmentation, and/or the like, to localize the individual cells present therein. Moreover, in some cases, the cell classification model may include a first machine learning model trained to identify, within the image of the biological sample, one or more visible features that are capable of being identified, localized, interpreted, inferred, and/or otherwise detected through a visual inspection of the image, for example, by a human, a machine, an algorithm, and/or the like. The cell classification may further include a second machine learning model trained to determine, based at least on the one or more visible features extracted from the image of the biological sample, one or more cell types present in the image. Alternatively and/or additionally, the cell classification model may be implemented as an end-to-end model that determines the cell types present in the image based on one or more hidden features extracted from the image, which may not necessarily correspond to the aforementioned visible features.

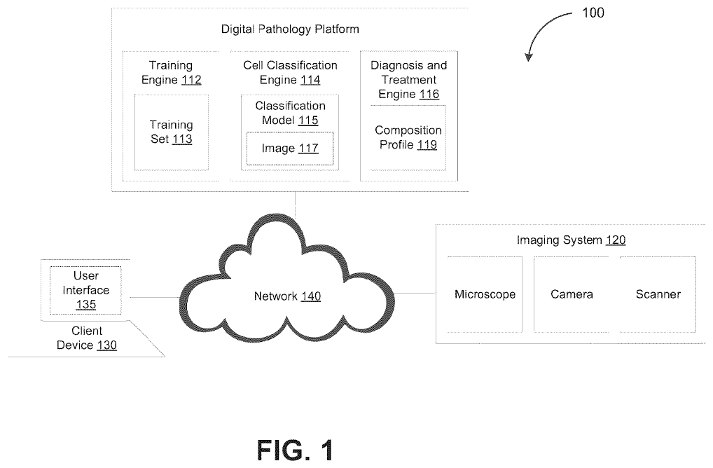

FIG. 1 depicts a system diagram illustrating an example of a digital pathology system 100, in accordance with some example embodiments. Referring to FIG. 1, the digital pathology system 100 may include a digital pathology platform 110, an imaging system 120, and a client device 130. As shown in FIG. 1, the digital pathology platform 110, the imaging system 120, and the client device 130 may be communicatively coupled via a network 140. The network 140 may be a wired network and/or a wireless network including, for example, a local area network (LAN), a virtual local area network (VLAN), a wide area network (WAN), a public land mobile network (PLMN), the Internet, and/or the like. The imaging system 120 may include one or more imaging devices including, for example, a microscope, a digital camera, a whole slide scanner, a robotic microscope, and/or the like. The client device 130 may be a processor-based device including, for example, a workstation, a desktop computer, a laptop computer, a smartphone, a tablet computer, a wearable apparatus, and/or the like.

Referring again to FIG. 1, the digital pathology platform 110 may include a training engine 112, a cell classification engine 114, and a diagnosis and treatment engine 116. As shown in FIG. 1, the cell classification engine 114 may include a cell classification model 115 trained to identify one or more cell types present in an image 117 depicting a biological sample. In some cases, the image 117 may be a stained whole slide image (WSI) including, for example, a hematoxylin and eosin (H&E) stained whole slide image, a multiplex immunofluorescence (MxIF) stained whole slide image, an immunohistochemical (IHC) stained whole slide image, and/or the like. Moreover, in some cases, the image 117 may have undergone segmentation, such as watershed cell segmentation, machine learning based cell segmentation, and/or the like, to localize the individual cells present therein. The cell classification model 115 may be trained to recognize a macrophage class, which includes foamy macrophages, intra-alveolar macrophages, and pigmented stromal macrophages, as well as a separate stromal cell class that includes non-pigmented stromal macrophages and fibroblasts. Furthermore, in some cases, the cell classification model 115 may be trained to recognize tumor cells, plasma cells, lymphocytes, endothelial cells, adipocytes, and neutrophils.

In some example embodiments, the training engine 112 may train, based at least on a training set 113, the cell classification model 115 to differentiate between a plurality of cell types including, for example, macrophages, stromal cells, tumor cells, plasma cells, lymphocytes, endothelial cells, adipocytes, and neutrophils. In some cases, the training engine 112 may generate the training set 113, which includes one or more images whose ground truth labels identifying the cell types present therein are determined based on expert annotations. The ground truth label assigned to an image may be determined based at least on the confidence value of the corresponding expert annotation. For example, where the expert annotation indicating the presence of a macrophage in the image is associated with a confidence value satisfying one or more thresholds (e.g., the probability of the expert annotation being accurate exceeds a threshold value), the image may be assigned a ground truth label indicating the presence of a macrophage in the image. Alternatively, where the expert annotation indicating the presence of a macrophage in the image is associated with a confidence value that fails to satisfy the one or more thresholds (e.g., the probability of the annotation being accurate fails to exceed the threshold value), the image may be assigned a ground truth label indicating the presence of a stromal cell in the image.

Where the image has been segmented to localize the individual cells present therein, the ground truth label may be assigned to one or more corresponding pixels. For instance, where the expert annotation indicating the presence of a macrophage in the image is associated with a confidence value satisfying one or more thresholds (e.g., the probability of the annotation being accurate exceeds a threshold value), the ground truth label assigned to the image may identify the one or more pixels corresponding to the macrophage. Alternatively, where the expert annotation indicating the presence of a macrophage in the image is associated with a confidence value that fails to satisfy the one or more thresholds (e.g., the probability of the annotation being accurate fails to exceed the threshold value), the ground truth label assigned to the image may identify the one or more pixels corresponding to the stromal cell.

In some example embodiments, the cell classification engine 114 may generate, based at least on the one or more cell types identified within the image 117, a composition profile 119 for the biological sample depicted in the image. For example, in some cases, the composition profile 119 for the biological sample may include one or more of the cell types, such as macrophages, stromal cells, lymphocytes, tumor cells, plasma cells, endothelial cells, adipocytes, and neutrophils, identified as present within the biological sample. Alternatively and/or additionally, the composition profile 119 for the biological sample may include a quantity, a relative proportion, a density, and/or a spatial distribution of the one or more cell types present in the biological sample.

In some example embodiments, the composition profile of the biological sample depicted in the image 117 may be generated to include an indication of whether cells identified as one cell type are present within a threshold distance of cells identified as another cell type. For example, in some cases, the composition profile of the biological sample may be generated to include an indication of whether cells identified as the second cell type are within a threshold distance of cells identified as lymphocytes. Alternatively and/or additionally, the composition profile of the biological sample depicted in the image 117 may be generated to include a density and/or a spatial distribution of one or more cell types across a tumor region and/or a non-tumor region of the biological sample. For instance, in some cases, the composition profile of the biological sample may be generated to include a first indication of whether cells identified as one cell type are present in a tumor region of the biological sample. Furthermore, in some cases, the composition profile of the biological sample may be generated to include a second indication of whether cells identified as another cell type are also present in the tumor region of the biological sample and/or a non-tumor region of the biological sample. Accordingly, in some cases, the composition profile of the biological sample is generated to include an indication of whether cells identified as the first cell type and/or the second cell type are present in a tumor region of the biological sample. Alternatively and/or additionally, the composition profile of the biological sample is generated to include an indication of whether cells identified as the first cell type and/or the second cell type are present in a non-tumor region of the biological sample.

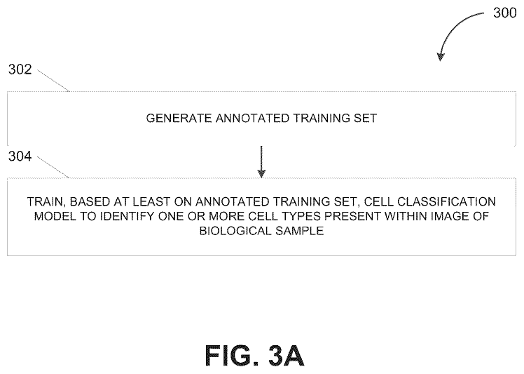

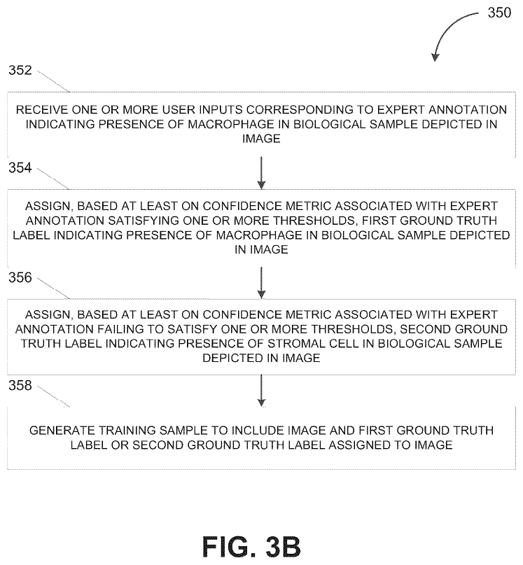

In some cases, the diagnosis and treatment engine 116 may determine, based at least on the composition profile 119 of the biological sample depicted in the image 117, at least one of a disease diagnosis, disease progression, disease burden, and treatment response for a patient associated with the biological sample. For instance, in some cases, the patient may be a non-small cell lung cancer (NSCLC) patient who is identified as a responder (or non-responder) to a combination immunotherapy, such as a T-cell immunoreceptor with Ig and ITIM domains (TIGIT) and atezolizumab combination therapy, based at least on the presence of macrophages within a threshold distance of lymphocytes (e.g., CD8 positive T-cells) in the biological sample. In this context, treatment response may be evaluated based on a variety of clinical outcomes including, for example, progression free survival, overall survival, mortality, and/or the like. To further illustrate, FIG. 3 depicts a schematic diagram illustrating an example of a tumor microenvironment populated by tumor cells, lymphocytes, stromal cells, macrophages, plasma cells, endothelial cells, adipocytes, and neutrophils. In some cases, proximity (e.g., less than 40-μm distance) between macrophages and lymphocytes (e.g., CD8 positive T-cells) within the tumor microenvironment may serve as a biomarker for differentiating between responders and non-responders to a combination immunotherapy, such as a T-cell immunoreceptor with Ig and ITIM domains (TIGIT) and atezolizumab combination therapy, particularly amongst the population of non-small cell lung cancer (NSCLC) patients whose cancer cells are PD-L1 negative or exhibit a low expression level of the PD-L1 gene.

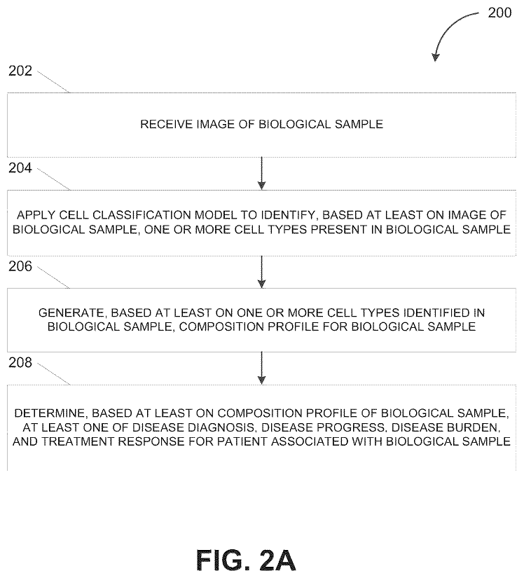

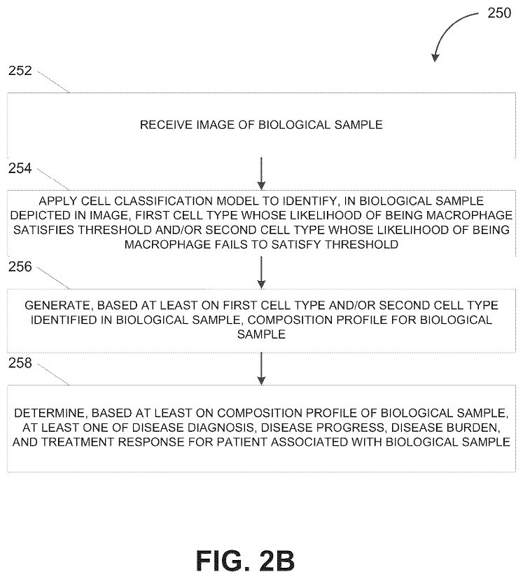

FIG. 2A depicts a flowchart illustrating an example of a process 200 for identifying one or more cell types present within an image of a biological sample, in accordance with some example embodiments. Referring to FIGS. 1-2A, the process 200 may be performed by the digital pathology platform 110, for example, by the cell classification engine 114 to identify, for example, the one or more cell types present within the image 117.

At 202, the digital pathology platform 110 may receive an image of a biological sample. For example, in some cases, the digital pathology platform 110 may receive, from the imaging system 120, the image 117. In some cases, the image 117 may be a whole slide image depicting a biological sample. Moreover, in some cases, the image 117 may be a stained whole slide image including, for example, a hematoxylin and eosin (H&E) stained whole slide image, a multiplex immunofluorescence (MxIF) stained whole slide image, an immunohistochemical (IHC) stained whole slide image, and/or the like.

At 204, the digital pathology platform 110 may apply a cell classification model to identify, based at least on the image of the biological sample, one or more cell types present in the biological sample. In some example embodiments, the digital pathology platform 110, for example, the cell segmentation engine 114 may apply the cell segmentation model 115 to determine, based at least on the image 117, one or more cell types present in the biological sample depicted in the image 117. The cell segmentation model 115 may be trained to differentiate between a variety of different cell types including, for example, a first cell type whose likelihood of being a macrophage satisfies a threshold and a second cell type whose likelihood of being a macrophage fails to satisfy the threshold. For example, in some cases, the cell segmentation model 115 may be trained to differentiate between a stromal cell class, which includes fibroblasts and non-pigmented stromal macrophages, and macrophage class that includes foamy macrophages, intra-alveolar macrophages, and pigmented stromal macrophages. Furthermore, in some cases, the cell segmentation model 115 may be trained to also differentiate between tumor cells, plasma cells, lymphocytes, endothelial cells, adipocytes, and neutrophils in addition to stromal cells and macrophages.

In some example embodiments, the cell classification model 115 may classify each cell depicted in the image 117 by at least assigning a label to each individual cell present in the biological sample depicted in the image 117. In some cases, the label assigned to each cell may be a “hard label” indicating a specific cell type. Alternatively, the cell classification model 115 may determine, for each cell, a “soft label,” which may be a probability distribution of the probabilities p₀, p₁, . . . , pₙ of the cell being positive for each of an n different cell types (e.g., stromal cell, macrophage, tumor cell, lymphocyte, and plasma cell).

In some example embodiments, the cell classification model 115 may operate on a segmented version of the image 117. That is, prior to the cell classification model 115 being applied to the image 117, the image 117 may undergo segmentation (e.g., watershed cell segmentation, machine learning based cell segmentation, and/or the like) to localize the individual cells present therein. Accordingly, in some cases, the labels assigned to the image 117 by the cell classification model 115 may include a label for one or more of the pixels identified through segmentation as being a part of a cell present in the image 117. For example, where the image 117 is segmented to localize one or more of the cells present therein, the cell classification model 115 may assign, to each pixel associated with a cell, a label indicating the corresponding cell type (e.g., macrophage, stromal cell, plasma cell, lymphocyte, or tumor cell).

In some example embodiments, the cell classification model 115 may be implemented using a variety of machine learning models including, for example, a gradient-boosted trees binary classifier, a random forest, a naïve Bayes classifier, a neural network, a k-means clustering model, a logistic regression model, and/or the like. In some cases, the cell classification model 115 may include a first machine learning model trained to identify, within the image 117 of the biological sample, one or more visible features, and a second machine learning model trained to identify the one or more cell types based at least on the one or more visible features extracted from the image 117. As used herein, the term “visible feature” may refer to a feature that is capable of being identified, localized, interpreted, inferred, and/or otherwise detected through a visual inspection of the image, for example, by a human, a machine, an algorithm, and/or the like. Alternatively and/or additionally, the cell classification model 115 may be implemented as an end-to-end model that determines the cell types present in the image 117 based on one or more hidden features extracted from the image 117, which may not necessarily correspond to the aforementioned visible features.

At 206, the digital pathology platform 110 may generate, based at least on the one or more cell types identified in the biological sample, a composition profile for the biological sample. In some example embodiments, the digital pathology platform 110, for example, the cell classification engine 114 may generate, based at least on the one or more cell types identified as present within the image 117, the composition profile 119 for the biological sample depicted in the image 117. For example, in some cases, the composition profile 119 may indicate the one or more cell types present in the biological sample including, for instance, stromal cells, macrophages, lymphocytes, plasma cells, tumor cells, endothelial cells, adipocytes, and neutrophils. Alternatively and/or additionally, the composition profile 119 may indicate one or more of a quantity, a relative proportion, a density, and a spatial distribution of the one or more cell types present within the image 117.

At 208, the digital pathology platform 110 may determine, based at least on the composition profile of the biological sample, at least one of a disease diagnosis, a disease progress, a disease burden, and a treatment response for a patient associated with the biological sample. In some example embodiments, the digital pathology platform 110, for example, the diagnosis and treatment engine 116 may determine, based at least on the composition profile 119 of the biological sample depicted in the image 117, at least one of a disease diagnosis, a disease progress, a disease burden, and a treatment response for a patient associated with the biological sample. In some cases, the composition profile 119 of the biological sample may be generated by the cell classification engine 114 to indicate one or more of the cell types present in the biological sample. Alternatively and/or additionally, the cell classification engine 114 may generate the composition profile 119 to indicate one or more of a quantity, a relative proportion, a density, and a spatial distribution of the one or more cell types in the biological sample. As shown in FIGS. 2A-B, for a non-small cell lung cancer (NSCLC) patient, the distance between macrophages and lymphocytes (e.g., CD8 positive T-cells) in the tumor microenvironment may be indicative of whether the patient is a responder (or non-responder) to combination immunotherapy such as the combination of T-cell immunoreceptor with Ig and ITIM domains (TIGIT) with atezolizumab.

FIG. 2B depicts a flowchart illustrating another example of a process 250 for identifying one or more cell types present within an image of a biological sample, in accordance with some example embodiments. Referring to FIGS. 1 and 2A-B, the process 250 may be performed by the digital pathology platform 110, for example, by the cell classification engine 114, to identify one or more macrophages and stromal cells present in the biological sample depicted in the image 117.

At 252, the digital pathology platform 110 may receive an image of a biological sample. For example, in some cases, the digital pathology platform 110 may receive, from the imaging system 120, the image 117. As noted, in some cases, the image 117 may be a whole slide image depicting a biological sample. Moreover, in some cases, the image 117 may be a stained whole slide image including, for example, a hematoxylin and eosin (H&E) stained whole slide image, a multiplex immunofluorescence (MxIF) stained whole slide image, an immunohistochemical (IHC) stained whole slide image, and/or the like.

At 254, the digital pathology platform 110 may apply a cell classification model to identify, in the biological sample depicted in the image, a first cell type whose likelihood of being a macrophage satisfies a threshold and/or a second cell type whose likelihood of being a macrophage fails to satisfy the threshold. In example embodiments, the digital pathology platform 110, for example, the cell segmentation engine 114 may apply the cell segmentation model 115 to determine, based at least on the image 117, one or more cell types present in the biological sample depicted in the image 117. In some cases, the cell segmentation model 115 may be trained to differentiate between macrophages and stromal cells. That is, the cell segmentation model 115 may be trained to identify, within the biological sample depicted in the image 117, a first cell type whose likelihood of being a macrophage satisfies a threshold and a second cell type whose likelihood of being a macrophage fails to satisfy the threshold. In some cases, the first cell type may correspond to a macrophage class that includes foamy macrophages, intra-alveolar macrophages, and pigmented stromal macrophages whereas the second cell type may correspond to a stromal cell class that includes fibroblasts and non-pigmented stromal macrophages. In some cases, the cell segmentation model 115 may be further trained to identify, within the biological sample depicted in the image 117, additional cell types including, for example, tumor cells, plasma cells, lymphocytes, endothelial cells, adipocytes, neutrophils, and/or the like.

At 256, the digital pathology platform 110 may generate, based at least on the first cell type and/or the second cell type identified in the biological sample, a composition profile for the biological sample. In some example embodiments, the digital pathology platform 110, for example, the cell classification engine 114 may generate the composition profile 119 of the biological sample depicted in the image 117 to include an indication of whether the first cell type and/or the second cell type are present in the biological sample. That is, in some cases, the composition profile 119 may be generated to include an indication of whether macrophages and/or stromal cells are present in the biological sample depicted in the image 117. In some cases, the cell classification engine 114 may generate the composition profile 119 to include one or more of a quantity, a relative proportion, a density, and a spatial distribution of the first cell type (e.g., macrophages) and/or the second cell type (e.g., stromal cells) within the biological sample depicted in the image 117. Alternatively and/or additionally, the cell classification engine 114 may generate the composition profile 119 to include a density and/or a spatial distribution of the first cell type (e.g., macrophages) and/or the second cell type (e.g., stromal cells) across a tumor region and/or a non-tumor region of the biological sample. For example, in some cases, the composition profile 119 of the biological sample may be generated to include a first indication of whether the first cell type (e.g., macrophages) and/or the second cell type (e.g., stromal cells) are present in a tumor region of the biological sample. Furthermore, in some cases the composition profile 119 of the biological sample may be generated to include a second indication of whether the first cell type (e.g., macrophages) and/or the second cell type (e.g., stromal cells) are present in a non-tumor region of the biological sample.

At 258, the digital pathology platform 110 may determine, based at least on the composition profile of the biological sample, at least one of a disease diagnosis, a disease progress, a disease burden, and a treatment response for a patient associated with the biological sample. In some example embodiments, the digital pathology platform 110, for example, the diagnosis and treatment engine 116 may determine, based at least on the composition profile 119 of the biological sample depicted in the image 117, at least one of a disease diagnosis, a disease progress, a disease burden, and a treatment response for a patient associated with the biological sample. For a non-small cell lung cancer (NSCLC) patient, for example, the distance between macrophages and lymphocytes (e.g., CD8 positive T-cells) in the tumor microenvironment may be indicative of whether the patient is a responder (or non-responder) to combination immunotherapy such as the combination of T-cell immunoreceptor with Ig and ITIM domains (TIGIT) with atezolizumab.

FIG. 3A depicts a flowchart illustrating an example of a process 300 for training a cell classification model, in accordance with some example embodiments. Referring to FIGS. 1 and 3, the process 300 may be performed by the digital pathology platform 110, for example, by the training engine 112, in order to train the cell classification model 115 to identify one or more cell types present within the biological sample depicted in the image 117.

At 302, the digital pathology platform 110 may generate an annotated training set. In some example embodiments, the digital pathology platform 110, for example, the training engine 112, may train the cell classification model 115 based at least on the training set 113. In some cases, the training set 113 may be an annotated training set in which each image of a biological sample is annotated with one or more ground truth labels of the cell types present in the biological sample. For example, where the images in the training set 113 are segmented to localize the individual cells present therein, each pixel that depicts a cell may be associated with a ground truth label indicating the corresponding cell type.

In some example embodiments, the training engine 112 may generate the training set by at least assigning, based at least on expert annotations, one or more ground truth labels to each image included in the training set 113. For example, in instances where the expert annotation indicating the presence of a macrophage is associated with a confidence value satisfying one or more thresholds (e.g., the probability of the annotation being accurate exceeds a threshold value), the training engine 112 may assign a ground truth label identifying one or more corresponding pixels as depicting a macrophage. Alternatively, in instances where the expert annotation indicating the presence of a macrophage is associated with a confidence value that fails to satisfy the one or more thresholds (e.g., the probability of the annotation being accurate fails to exceed the threshold value), the training engine 112 may assign a ground truth label identifying one or more corresponding pixels as depicting a stromal cell. In doing so, the cell classification engine 115 may be trained to differentiate between a macrophage class that includes foamy macrophages, intra-alveolar macrophages, and pigmented stromal macrophages and a stromal cell class that includes fibroblasts and non-pigmented stromal macrophages. Non-pigmented stromal macrophages may be included in a separate stromal cell class along with fibroblasts in order to account for the uncertainty associated with expert annotations. That is, non-pigmented stromal macrophages tend to be visually confounded with fibroblasts. As such, the expert annotations associated with non-pigmented stromal macrophages are not sufficiently reliable for training the cell classification model 115 to recognize non-pigmented stromal macrophages in a single macrophage class along with the other types of macrophages, such as foamy macrophages, intra-alveolar macrophages, and pigmented stromal macrophages, that can identified with a much greater degree of certainty. The performance of the cell classification model 115, particularly with respect to the identification of those macrophages with high confidence expert annotations, may therefore be improved by training the cell classification model 115 to recognize non-pigmented stromal macrophages as a part of a separate stromal cell class that also includes fibroblasts.

In a conventional paradigm, the one or more images of biological samples forming the training set 113 would be annotated based on a single monolithic class of macrophages that includes the foamy macrophages, intra-alveolar macrophages, and pigmented stromal macrophages that are associated with high-confidence expert annotations as well as the non-pigmented stromal macrophages that are associated with low-confidence expert annotations. That fibroblasts are often confounded with non-pigmented stromal macrophages but are a part of a separate fibroblast class means that training the cell classification model 115 to recognize cancer cells, lymphocytes, fibroblasts, plasma cells, and macrophages without differentiating between high-confidence macrophages and low-confidence macrophages may perpetuate the error present in the insufficiently reliable expert annotations associated with fibroblasts and non-pigmented stromal macrophages.

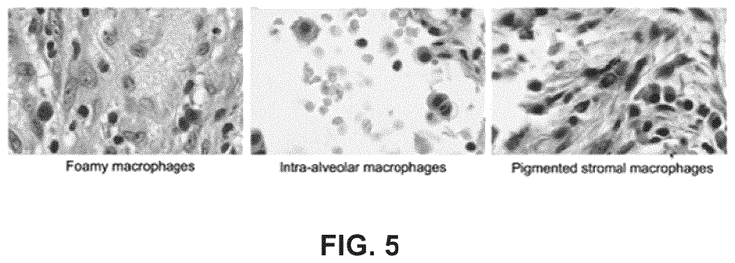

Instead of the five cell types, the cell classification model 115 may be trained to differentiate between stromal cells, macrophages, lymphocytes, plasma cells, tumor cells, endothelial cells, adipocytes, and neutrophils. In particular, the cell classification model 115 may be trained to recognize the non-pigmented stromal macrophages that are associated with low-confidence expert annotations as a part of a separate stromal cell class along with fibroblasts while the foamy macrophages, intra-alveolar macrophages, and pigmented stromal macrophages that are associated with high-confidence expert annotations are part of the macrophage class. FIG. 5 shows the three types of macrophages (e.g., foamy macrophages, intra-alveolar macrophages, and pigmented stromal macrophages) that are identifiable with a confidence value satisfying one or more thresholds (e.g., a probability of being accurate that exceeds a threshold value). Training the cell classification model 115 to recognize a macrophage class that includes foamy macrophages, intra-alveolar macrophages, and pigmented stromal macrophages but not non-pigmented stromal macrophages may prevent the uncertainty in the expert annotations associated with non-pigmented stromal macrophages from diminishing the performance of the cell classification model 115 in identifying foamy macrophages, intra-alveolar macrophages, and pigmented stromal macrophages.

At 304, the digital pathology platform 110 may train, based at least on the annotated training set, a cell classification model to identify one or more cell types present within an image of a biological sample. In some example embodiments, the digital pathology platform 110, for example, the training engine 112 may train the cell classification model 115 based at least on the training set 113. As noted, the cell classification model 115 may be trained to differentiate between a plurality of cell types that includes stromal cells, macrophages, plasma cells, lymphocytes, tumor cells, endothelial cells, adipocytes, and neutrophils. Moreover, the training of the cell classification model 115 may include adjusting the learnable parameters of the cell classification model 115 until reaching a convergence in which the loss in the output of the cell classification model 115 settles to a certain error range. For example, in some cases, the training of the cell classification model 115 may include adjusting one or more of the weights and biases of the cell classification model 115 through a backward propagation of a loss present in the output of the cell classification model 115. In this context, the loss in the output of the cell classification model 115 may be a quantity corresponding to a discrepancy between the labels (e.g., cell type labels) assigned by the cell classification model 115 to an image of a biological sample and the ground truth labels (e.g., ground truth cell types) associated with the image.

FIG. 3B depicts a flowchart illustrating an example of a process 350 for generating a training set for training a cell classification model, in accordance with some example embodiments. Referring to FIGS. 1 and 3A-B, the process 350 may be performed by the digital pathology platform 110, for example, by the training engine 112, in order to generate the training set 113 for training the cell classification model 115 to identify one or more cell types present within the biological sample depicted in the image 117. In some cases, the process 350 may be performed to implement operation 302 of the process 300 shown in FIG. 3A.

At 352, the digital pathology platform 110 may receive one or more user inputs corresponding to an expert annotation indicating a presence of a macrophage in a biological sample depicted in an image. In some example embodiments, the digital pathology platform 110, for example, the training engine 112 may receive, from the client device 130, one or more user inputs corresponding to an expert annotation indicating the presence of a macrophage in a biological sample depicted in an image. For example, in some cases, the expert annotation may identify one or more pixels in the image as depicting a macrophage.

At 354, the digital pathology platform 110 may assign, based at least on a confidence metric associated with the expert annotation satisfying one or more thresholds, a first ground truth label indicating the presence of a macrophage in the biological sample depicted in the image. In some example embodiments, the digital pathology platform 110, for example, the training engine 112, may assign a first ground truth label to the image if the confidence metric associated with the expert annotation satisfies one or more thresholds. For example, in some cases, where the likelihood of the expert annotation accurately identifying the macrophage exceeds a threshold value, the training engine 112 may assign the first ground truth label to the image. In some cases, the first ground truth label may identify one or more corresponding pixels in the image as depicting a macrophage (e.g., foamy macrophages, intra-alveolar macrophages, and pigmented stromal macrophages).

At 356, the digital pathology platform 110 may assign, based at least on the confidence metric associated with the expert annotation failing to satisfy the one or more thresholds, a second ground truth label indicating the presence of a stromal cell in the biological sample depicted in the image. In some example embodiments, the digital pathology platform 110, for example, the training engine 112, may assign a second ground truth label to the image if the confidence metric associated with the expert annotation fails to satisfy one or more thresholds. For example, in some cases, where the likelihood of the expert annotation accurately identifying the macrophage fails to exceed the threshold value, the training engine 112 may assign the second ground truth label to the image. In some cases, the second ground truth label may identify one or more corresponding pixels in the image as depicting a stromal cell (e.g., fibroblasts and non-pigmented stromal macrophages).

At 358, the digital pathology platform 110 may generate a training sample including the image of the biological sample and the first ground truth label or the second ground truth label assigned to the image. In some example embodiments, the digital pathology platform 110 may generate, for inclusion in the training set 113, a training sample that includes the image of the biological sample and either the first ground truth label or the second ground truth label assigned to the image.

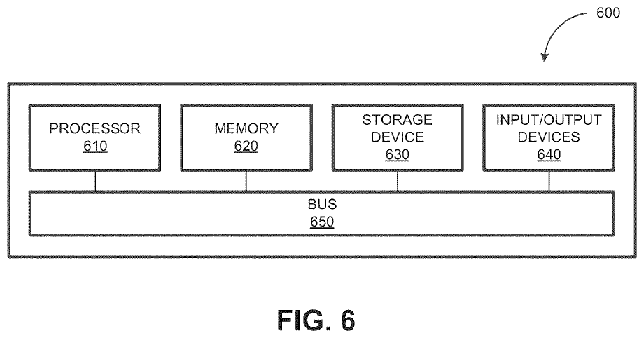

FIG. 6 depicts a block diagram illustrating an example of computing system 600, in accordance with some example embodiments. Referring to FIGS. 1 and 6, the computing system 600 may be used to implement the digital pathology platform 110, the imaging system 120, the client device 130, and/or any components therein.

As shown in FIG. 6, the computing system 600 can include a processor 610, a memory 620, a storage device 630, and an input/output device 640. The processor 610, the memory 620, the storage device 630, and the input/output device 640 can be interconnected via a system bus 650. The processor 610 is capable of processing instructions for execution within the computing system 600. Such executed instructions can implement one or more components of, for example, the digital pathology platform 110, the imaging system 120, the client device 130, and/or the like. In some example embodiments, the processor 610 can be a single-threaded processor. Alternately, the processor 610 can be a multi-threaded processor. The processor 610 is capable of processing instructions stored in the memory 620 and/or on the storage device 630 to display graphical information for a user interface provided via the input/output device 640.

The memory 620 is a computer readable medium such as volatile or non-volatile that stores information within the computing system 600. The memory 620 can store data structures representing configuration object databases, for example. The storage device 630 is capable of providing persistent storage for the computing system 600. The storage device 630 can be a floppy disk device, a hard disk device, an optical disk device, or a tape device, or other suitable persistent storage means. The input/output device 640 provides input/output operations for the computing system 600. In some example embodiments, the input/output device 640 includes a keyboard and/or pointing device. In various implementations, the input/output device 640 includes a display unit for displaying graphical user interfaces.

According to some example embodiments, the input/output device 640 can provide input/output operations for a network device. For example, the input/output device 640 can include Ethernet ports or other networking ports to communicate with one or more wired and/or wireless networks (e.g., a local area network (LAN), a wide area network (WAN), the Internet).

In some example embodiments, the computing system 600 can be used to execute various interactive computer software applications that can be used for organization, analysis and/or storage of data in various formats. Alternatively, the computing system 600 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 640. The user interface can be generated and presented to a user by the computing system 600 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

### EXAMPLE EMBODIMENTS

Embodiments disclosed herein may include:
1. A computer-implemented method, comprising:

receiving an image of a biological sample;

applying a cell classification model to identify, based at least on the image of the biological sample, one or more cell types present in the biological sample, the cell classification model being trained to differentiate between a plurality of cell types including a first cell type whose likelihood of being a macrophage satisfies a threshold and a second cell type whose likelihood of being the macrophage fails to satisfy the threshold; and

generating, based at least on the one or more cell types identified in the biological sample, a composition profile for the biological sample.
2. The method of embodiment 1, wherein the first cell type is macrophages.
3. The method of embodiment 1 or embodiment 2, wherein the first cell type includes foamy macrophages, intra-alveolar macrophages, and pigmented stromal macrophages.
4. The method of any one of embodiments 1-3, wherein the second cell type is stromal cells.
5. The method of any one of embodiments 1-4, wherein the second cell type includes fibroblasts and non-pigmented stromal macrophages.
6. The method of any one of embodiments 1-5, wherein the plurality of cell types further include tumor cells, lymphocytes, plasma cells, endothelial cells, adipocytes, and neutrophils.
7. The method of any one of embodiments 1-6, wherein the cell classification model includes a first machine learning model trained to extract one or more features from the image of the biological sample, and wherein the cell classification model further includes a second machine learning model trained to identify, based at least on the one or more features extracted from the image of the biological sample, the one or more cell types present in the biological sample.
8. The method of any one of embodiments 1-7, wherein the cell classification model includes an end-to-end machine learning model trained to identify the one or more cell types present in the biological sample.
9. The method of any one of embodiments 1-8, wherein the image is a whole slide image.
10. The method of any one of embodiments 1-9, wherein the image is a hematoxylin and eosin (H&E) stained whole slide image or an immunohistochemical (IHC) stained whole slide image.
11. The method of any one of embodiments 1-10, wherein the biological sample includes one or more tissue fragments, free cells, and/or body fluids.
12. The method of any one of embodiments 1-11, wherein the biological sample includes tumor tissue.
13. The method of any one of embodiments 1-12, wherein the composition profile of the biological sample is generated to include a density and/or a spatial distribution of the one or more cell types present in the biological sample.
14. The method of any one of embodiments 1-13, wherein the composition profile of the biological sample is generated to include an indication of whether cells identified as one cell type are present within a threshold distance of cells identified as another cell type.
15. The method of any one of embodiments 1-14, wherein the composition profile of the biological sample is generated to include an indication of whether cells identified as the second cell type are within a threshold distance of cells identified as lymphocytes.
16. The method of any one of embodiments 1-15, wherein the composition profile of the biological sample is generated to include a quantity and/or a relative proportion of the one or more cell types present in the biological sample.
17. The method of any one of embodiments 1-16, wherein the composition profile of the biological sample is generated to include a first indication of whether cells identified as one cell type are present in a tumor region of the biological sample.
18. The method of embodiment 17, wherein the composition profile of the biological sample is further generated to include a second indication of whether cells identified as another cell type are also present in the tumor region of the biological sample and/or a non-tumor region of the biological sample.
19. The method of any one of embodiments 1-18, wherein the composition profile of the biological profile is generated to include a spatial distribution of the one or more cell types across a tumor region of the biological sample and/or a non-tumor region of the biological sample.
20. The method of any one of embodiments 1-19, wherein the composition profile of the biological sample is generated to include an indication of whether cells identified as the first cell type and/or the second cell type are present in a tumor region of the biological sample.
21. The method of any one of embodiments 1-20, wherein the composition profile of the biological sample is generated to include an indication of whether cells identified as the first cell type and/or the second cell type are present in a non-tumor region of the biological sample.
22. The method of any one of embodiments 1-21, further comprising:

training, based at least on training data, the cell classification model to identify the one or more cell types present in the biological sample.
23. The method of embodiment 22, further comprising:

generating the training data to include one or more images annotated with ground truth labels identifying at least one cell type present in each image.
24. The method of embodiment 23, wherein the generating of the training data includes assigning, to the one or more images, a first ground truth label identifying the first cell type based at least on a first expert annotation identifying one or more macrophages being associated with a first confidence value that satisfies one or more thresholds.
25. The method of embodiment 24, wherein the generating of the training data further includes assigning, to the one or more images, a second ground truth label identifying the second cell type based at least on a second expert annotation identifying the one or more macrophages being associated with a second confidence value that fails to satisfy the one or more thresholds.
26. The method of any one of embodiments 1-25, further comprising:

determining, based at least on the composition profile of the biological sample, at least one of a disease diagnosis, a disease progress, a disease burden, and a treatment response for a patient associated with the biological sample.
27. The method of any one of embodiments 1-26, further comprising:

identifying, based at least on the composition profile of the biological sample, a patient associated with the biological sample as being a responder to a treatment or a non-responder to the treatment.
28. The method of any one of embodiments 1-27, further comprising:

determining, based at least on the composition profile of the biological sample, (i) a first likelihood of a patient associated with the biological sample responding to a treatment, (ii) a second likelihood of the patient relapsing after the treatment, and/or (iii) a durability of the patient's response to the treatment.
29. A system, comprising:

at least one data processor; and

at least one memory storing instructions, which when executed by the at least one data processor, result in operations comprising the method of any one of embodiments 1 to 28.
30. A non-transitory computer readable medium storing instructions, which when executed by at least one data processor, result in operations comprising the method of any one of embodiments 1 to 28.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A computer-implemented method, comprising:
receiving an image of a biological sample;
applying a cell classification model to identify, based at least on the image of the biological sample, one or more cell types present in the biological sample,
the cell classification model being trained to differentiate between a plurality of cell types including,
a first cell type whose likelihood of being a macrophage satisfies a threshold, and
a second cell type whose likelihood of being the macrophage fails to satisfy the threshold; and
generating, based at least on the one or more cell types identified in the biological sample, a composition profile for the biological sample.

2. The method of claim 1, wherein the first cell type is macrophages.

3. The method of claim 1, wherein the first cell type includes foamy macrophages, intra-alveolar macrophages, and pigmented stromal macrophages.

4. The method of claim 1, wherein the second cell type is stromal cells.

5. The method of claim 1, wherein the second cell type includes fibroblasts and non-pigmented stromal macrophages.

6. The method of claim 1, wherein the plurality of cell types further include tumor cells, lymphocytes, plasma cells, endothelial cells, adipocytes, and neutrophils.

7. The method of claim 1, wherein the cell classification model includes a first machine learning model trained to extract one or more features from the image of the biological sample, and wherein the cell classification model further includes a second machine learning model trained to identify, based at least on the one or more features extracted from the image of the biological sample, the one or more cell types present in the biological sample.

8. The method of claim 1, wherein the cell classification model includes an end-to-end machine learning model trained to identify the one or more cell types present in the biological sample.

9. The method of claim 1, wherein the biological sample includes tumor tissue.

10. The method of claim 1, wherein the composition profile of the biological sample is generated to include a density and/or a spatial distribution of the one or more cell types present in the biological sample.

11. The method of claim 1, wherein the composition profile of the biological sample is generated to include an indication of whether cells identified as one cell type are present within a threshold distance of cells identified as another cell type.

12. The method of claim 1, wherein the composition profile of the biological sample is generated to include an indication of whether cells identified as the second cell type are within a threshold distance of cells identified as lymphocytes.

13. The method of claim 1, wherein the composition profile of the biological sample is generated to include a quantity and/or a relative proportion of the one or more cell types present in the biological sample.

14. The method of claim 1, wherein the composition profile of the biological sample is generated to include a first indication of whether cells identified as one cell type are present in a tumor region of the biological sample.

15. The method of claim 1, wherein the composition profile of the biological profile is generated to include a spatial distribution of the one or more cell types across a tumor region of the biological sample and/or a non-tumor region of the biological sample.

16. The method of claim 1, wherein the composition profile of the biological sample is generated to include an indication of whether cells identified as the first cell type and/or the second cell type are present in a tumor region of the biological sample.

17. The method of claim 1, wherein the composition profile of the biological sample is generated to include an indication of whether cells identified as the first cell type and/or the second cell type are present in a non-tumor region of the biological sample.

18. The method of claim 1, further comprising:
determining, based at least on the composition profile of the biological sample, at least one of a disease diagnosis, a disease progress, a disease burden, and a treatment response for a patient associated with the biological sample.

19. The method of claim 1, further comprising:
identifying, based at least on the composition profile of the biological sample, a patient associated with the biological sample as being a responder to a treatment or a non-responder to the treatment.

20. The method of claim 1, further comprising:
determining, based at least on the composition profile of the biological sample, (i) a first likelihood of a patient associated with the biological sample responding to a treatment, (ii) a second likelihood of the patient relapsing after the treatment, and/or (iii) a durability of the patient's response to the treatment.

21. A system, comprising:
at least one data processor; and
at least one memory storing instructions, which when executed by the at least one data processor, result in operations comprising:
receiving an image of a biological sample;
applying a cell classification model to identify, based at least on the image of the biological sample, one or more cell types present in the biological sample,
the cell classification model being trained to differentiate between a plurality of cell types including,
a first cell type whose likelihood of being a macrophage satisfies a threshold, and
a second cell type whose likelihood of being the macrophage fails to satisfy the threshold; and
generating, based at least on the one or more cell types identified in the biological sample, a composition profile for the biological sample.

22. A non-transitory computer readable medium storing instructions, which when executed by at least one data processor, result in operations comprising:
receiving an image of a biological sample;
applying a cell classification model to identify, based at least on the image of the biological sample, one or more cell types present in the biological sample,
the cell classification model being trained to differentiate between a plurality of cell types including,
a first cell type whose likelihood of being a macrophage satisfies a threshold, and
a second cell type whose likelihood of being the macrophage fails to satisfy the threshold; and
generating, based at least on the one or more cell types identified in the biological sample, a composition profile for the biological sample.
